(19) United States
(12) Patent Application Publication
(10) Pub. No.: US 2012/0136267 A1
(43) Pub. Date: May 31, 2012

(54) Apparatus and Method For Visually Determining Whether Respiration is Occurring

(76) Inventors: Steven J. Derrick, West Mifflin, PA (US); Joyce A. Derrick, West Mifflin, PA (US)

(21) Appl. No.: 13/304,065

(22) Filed: Nov. 23, 2011

Related U.S. Application Data
(60) Provisional application No. 61/458,546, filed on Nov. 26, 2010.

(51) Int. Cl. A61B 5/08; A61M 16/04; A61M 16/00

(52) U.S. Cl. 600/529

(57) ABSTRACT

What is presented is an apparatus for visually determining whether respiration is occurring comprising a strip of material having a first side and a second side in which the first side has a layer of thermally sensitive material which undergoes a color change when exposed to a temperature variance caused by a subject's respiration. The thermally sensitive material could be a thermochromic liquid crystal compound. The second side could have a layer of adhesive which could be used to adhere the strip of material in the vicinity of the subject's upper lip. The strip of material could also be adhered to a visible surface of a respiratory assistance device with the adhesive.

This application takes priority from U.S. Provisional Application No. 61/458,546 which is hereby incorporated by reference.

### BACKGROUND

There is a need to quickly and easily determine whether or not a subject is in fact breathing both in medical emergencies and in routine medical situations. In emergency triage, there is a need for emergency medical technicians to quickly and easily determine whether or not a particular subject is beyond their ability to help so that their skill may be applied to subjects who they can treat. In hospice situations or in intensive care units, it is necessary to find out whether or not a subject is still respiring, especially where the subject's respiration is low or shallow and where placing a mirror under the subject's nose is an insufficient indicator of respiration.

Thermally sensitive materials including thermochromic liquid crystals (hereinafter “TLC”) which change color in response to temperature variations have been long known to have many medical and pharmacological uses such as measuring the temperature of a patient or ensuring that a particular drug is maintained at a required temperature level or noting whether or not a particular device is operating at a particular temperature range but there have not been any applications which address the need for monitoring of a subject's respiration

### SUMMARY

What is presented is an apparatus for visually determining whether respiration is occurring comprising a strip of material having a first side and a second side in which the first side has a layer of thermally sensitive material which undergoes a color change when exposed to a temperature variance caused by a subject's respiration. The thermally sensitive material could be a TLC compound. The second side could have a layer of adhesive which could be used to adhere the strip of material in the vicinity of the subject's upper lip. The strip of material could also be adhered to a visible surface of a respiratory assistance device with the adhesive. Examples of respiratory assistance devices include nasal cannula, oxygen delivery devices, ventilatory assistance devices such as laryngeal mask airways (LMAs), oral airways, or nasal airways, or endotracheal tubes.

The strip of material could be shaped like a T or any other size and shape to fit under the subject's nose and extend over the subject's lip. The strip of material could be made of paper, plastic, metal, cloth, thermoplastic polymers, silastic thermopolymers, polyurethanes, or any other suitable material or combination thereof.

In other embodiments, the strip of material could be permanently affixed to a respiratory assistance device. Such respiratory assistance devices could be manufactured to have a. layer of thermally sensitive material installed. The thermally sensitive material could be painted or sprayed onto the nasal cannula or be integrally formed into at least one visible surface of the respiratory assistance device during manufacture.

A method for visually determining whether respiration is occurring is also presented. According to the method, a strip of material having a first side and a second side is used in which the first side has a layer of thermally sensitive material which undergoes a color change when exposed to a temperature variance. The strip of material is placed so that it is sufficiently exposed to the expected region of respiration of a subject. Respiration is confirmed by observing whether the thermally sensitive material undergoes a color change. If the second side has a layer of adhesive, the strip of material could be adhered under the subject's nose or to a respiratory assistance device for extended observation of respiration.

Those skilled in the art will realize that this invention is capable of embodiments that are different from those shown and that the details of the devices and methods can be changed in various manners without departing from the scope of this invention. Accordingly, the drawings and descriptions are to be regarded as including such equivalent embodiments as do not depart from the spirit and scope of this invention.

### BRIEF DESCRIPTION OF DRAWINGS

For a more complete understanding and appreciation of this invention, and its many advantages, reference will be made to the following detailed description taken in conjunction with the accompanying drawings:

FIG. 1 is a view of an embodiment of the strip of material;

FIG. 2 shows a side view of the strip of material;

FIG. 3 shows a perspective view of the strip of material suspended in the vicinity of a subject's upper lip;

FIG. 4 shows a close up of the strip of material of FIG. 3;

FIG. 5 shows a color change occurring on the layer of thermally sensitive material of the strip of material of FIG. 4 when exposed to a temperature variance caused by a subject's nasal respiration;

FIG. 6 shows a color change occurring on the layer of thermally sensitive material of the strip of material of FIG. 4 when exposed to a temperature variance caused by a subject's oral respiration;

FIG. 7 shows an embodiment of the strip of material affixed in the vicinity of a subject's upper lip by the layer of adhesive;

FIG. 8 shows an alternative embodiment of the strip of material that is shaped to sufficiently cover the front of a nasal cannula;

FIG. 9 shows a side view of the strip of material of FIG. 8;

FIG. 10 shows the strip of material of FIG. 8 affixed to a nasal cannula;

FIG. 11 shows a side view of the strip of material of FIG. 10;

FIG. 12 shows an embodiment of the strip of material of FIG. 8 affixed to a different nasal cannula;

FIG. 13 shows a side view of the strip of material of FIG. 12; and

FIG. 14 shows a perspective view of the strip of material affixed to a nasal cannula installed on a subject.

### DETAILED DESCRIPTION

Referring to the drawings, some of the reference numerals are used to designate the same or corresponding parts through several of the embodiments and figures shown and described. Corresponding parts are denoted in different embodiments with the addition of lowercase letters. Variations of corresponding parts in form or function that are depicted in the figures are described. It will be understood that variations in the embodiments can generally be interchanged without deviating from the invention.

The apparatus presented herein provides a quick, simple, easily transported, and inexpensive way to determine whether respiration is occurring in a subject. The apparatus may be of use in a variety of situations. For example after an accident or in an emergency triage where medical technicians need to quickly and easily determine whether a subject is breathing. In other non-emergency circumstances such as in intensive care units, hospice care, etc. it is necessary to have a non-invasive means to determine whether a subject is breathing. While the apparatus is generally designed for use on human subjects, it may also be used in veterinary settings, on any animal where detecting the presence of respiration is required.

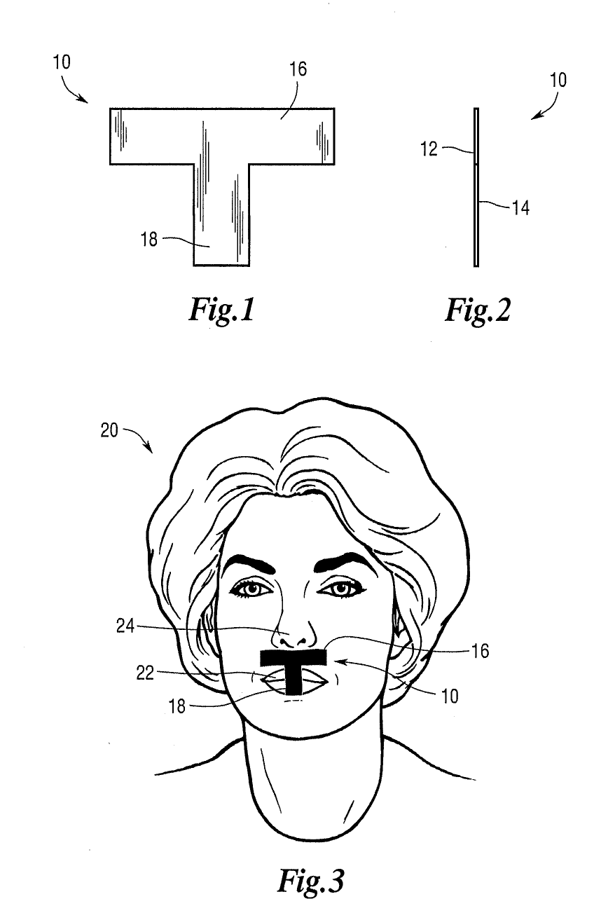

The preferred embodiment of the apparatus for visually determining whether respiration is occurring is best understood by comparing FIGS. 1, 2, and 3. The apparatus comprises a strip of material 10 that has a first side 12 and a second side 14. Typically the strip of material 10 is made from paper, plastic, metal, cloth, thermoplastic polymers (like polytetrafluoroethylene (PTFE) Teflon or isomers and variations thereof), silastic thermopolymers, polyurethanes, or any other suitable material or a combination of any of the above; although any material strong enough to withstand the normal application of the apparatus will be sufficient.

The first side 12 of the strip of material 10 has a layer of thermally sensitive material applied to it. This thermally sensitive material is designed to undergo a color change after exposure to a temperature variance from the respiration of a subject 20. The thermally sensitive material can be applied to the first side 12 of the strip of material 10 by any of a variety of ways. It is typically sprayed on the first side 12 of the strip of material 10 during the manufacturing process. However, it can also be applied by a brush, roller, stamp, or any other adequate method if the manufacturer so chooses.

The thermally sensitive material is designed to be colorimetrically reactive within a pre-selected and customizable temperature range (bandwidth). The thermally sensitive material is normally in a dormant state and will colorimetrically react within the selected temperature bandwidth. While in the dormant state, the vast majority of the thermally sensitive material may be one of several colors but is typically black in color. When the temperature of the thermally sensitive material is either raised or lowered outside the dormant state range, it will undergo a color change into colors that are not black. Usually, but not always, the thermally sensitive material will go through a range of varying color changes including those of the visible light spectrum: red, orange, yellow, green, blue, violet, or vice versa. In the various embodiments of the invention disclosed herein, a colorimetric reaction by the thermally sensitive material generally signals the presence of respiration in a subject.

While a temperature bandwidth between 27 and 32 degrees Celsius is generally chosen as the reactive state, the thermally sensitive material may also be designed during manufacture to be reactive at a different preselected temperature bandwidth. This can be done by designing the range of the reactive state of the thermally sensitive material to be at a higher or lower temperature. Preselecting the range of temperatures optimizes performance of the apparatus so an observer can determine whether there is respiration in different settings and circumstances, and under various environmental conditions. The apparatus can be used in both indoor/outdoor situations or in the most extreme environments such as an arid desert, a rainforest, the arctic, even emergency fire situations, etc. Preselecting this range also makes the apparatus perform in situations that do not occur in the most ideal environments and the person applying it does not have time to understand how their environment affects the color change (e.g. military personnel under the stresses of combat or EMS personnel arriving at the scene of an accident).

The thermally sensitive material may not solely be made from TLC, but where a TLC is used, there should be at least enough TLC to create a noticeable color change when exposed to temperature outside the dormant range. It should also be noted that any thermally sensitive material, not just ones comprising a TLC, that can change color for the general purposes of the apparatus could work just as well.

It is preferred that the thermally sensitive material is made from a TLC based compound. The Nematic LC Sprayable Coating manufactured by LCR Hallcrest, LLC is the TLC compound preferred for the apparatus in this embodiment.

As shown in FIG. 1, the strip of material 10 is preferably formed in a T-like shape. When the apparatus is located below the subject's nose 24 the horizontally elongated wings 16 span under the nostrils of the subject's nose 24 and the vertically elongated wing 18 extends over the subject's upper lip 22. Shaping the strip of material 10 like a T allows the apparatus to extend under the nasal and oral respiration pathways while requiring less material to be used in the manufacture of the apparatus.

As shown in FIGS. 3 through 6, to properly use the preferred embodiment of the apparatus, the strip of material 10 is placed, held, or otherwise suspended on the subject in the vicinity of the subject's upper lip 22 so that the second side 14 faces the subject 20 and the first side 12 faces outward so that a color change in the thermally sensitive material can be observed. The strip of material 10 is exposed to the expected regions of respiration: generally both nasal and oral respiration. Loose, but secure, attachment of the strip of material 10 is important because both the first side 12 and the second side 14 of the strip of material 10 needs to be exposed to as much respiratory flow as possible. As the subject's 20 body heat will induce a colorimetric reaction in the thermally sensitive material, it is important to keep at least the portion of the thermally sensitive material that is expected to be exposed to the subject's 20 respiration away from the subject's 20 skin. The strip of material 10 could be held in place by a technician so long as the technician is aware of any thermal effect imparted by the technician's fingers. Any other means of attachment to the subject 20 may be used, such as clips, hooks, elastic, extra lengths of material able to be tied off or otherwise secured, etc. but it is understood that such means are not strictly necessary for the functioning of the apparatus.

Typically the strip of material 10 is applied so that the horizontally elongated wings 16 sit below the subject's nose 24 and the vertically elongated wing 18 extends freely over the subject's upper lip 22. After the strip of material 10 is adequately secured to the subject, respiration is observed based on any color change occurring with the thermally sensitive material. The subject's 20 respiration about or around both the first side 12 and the second side 14 causes a temperature variance that is conducted around and through the strip of material 10 that the thermally sensitive material reacts to and changes color. The actual thermal flow occurs about both the first side 12 and the second side 14 as the temperature variance oscillates between inspiration and expiration.

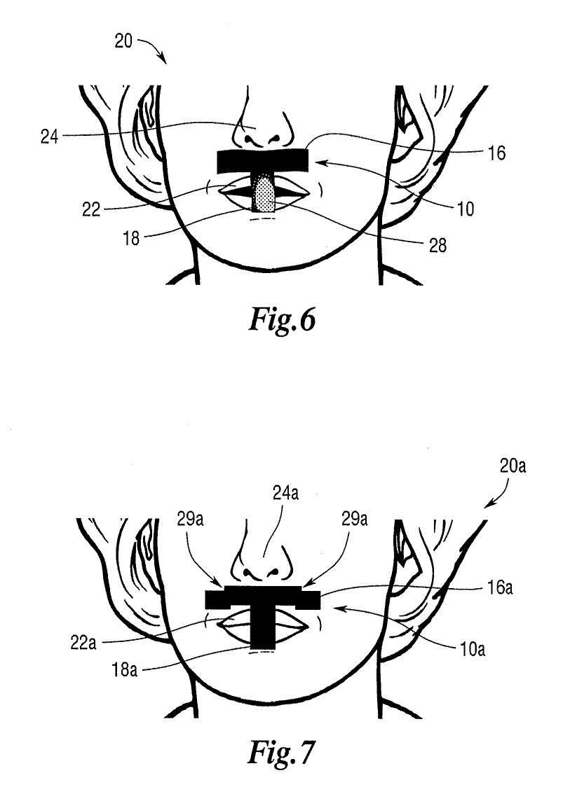

Thermal variations due to nasal respiration would be indicated by a color change 26 in the thermally sensitive material on the horizontally elongated wings 16 as shown in FIG. 5. Thermal variations due to oral respiration would be indicated by a color change 28 in the vertically elongated wing 18 as shown in FIG. 6. The thermally sensitive material remains dormant in those areas that are not exposed to the subject's nasal or oral respiration. A color change around the nostrils or just over the mouth would most likely be sufficient for the average observer to determine that the subject 20 is respiring. Even though the T shape is convenient, any form or shape of the strip of material 10 that can be sufficiently exposed to the expected region of a subject's 20 respiration may be used.

Some embodiments of the apparatus may have a layer of adhesive applied to the second side 14a of the strip of material 10a in order to be able to affix the strip of material 10a to the vicinity of the subject's 20a upper lip 22a, as shown for example in FIG. 7. Loose, but secure, attachment of the strip of material 10a is important because both the first side 12a and the second side 14a of the strip of material 10a needs to be exposed to as much respiratory flow as possible. As the subject's 20a body heat will induce a colorimetric reaction in the thermally sensitive material, it is important to keep at least the portion of the thermally sensitive material that is expected to be exposed to the subject's 20 respiration away from the subject's 20a skin. The strip of material 10a can be folded into creases 29a and secured at two points with the adhesive on the second side 14b to suspend the strip of material 10a under the subject's nose 24a as shown in FIG. 7.

Just as with the thermally sensitive material, the adhesive can be applied in a number of ways. Typically the adhesive would be sprayed on the second side 14b of the strip of material 10b during the manufacturing process. However, the adhesive can also be applied by a brush, roller, stamp, or any other adequate method that the manufacturer chooses.

The apparatus may be affixed directly to a subject or it may be attached to a respiratory assistance device such as a nasal cannula, an oxygen delivery device, a ventilatory assistance device such as a laryngeal mask airway (LMAs), an oral airway, or a nasal airway, an endotracheal tube, or any such product or device available in the industry. As when the apparatus is used directly on a subject, the apparatus may be affixed by any means such as clips, hooks, elastic, extra lengths of material able to be tied off or otherwise secured, etc. The preferred method would be to use a layer of adhesive as with earlier embodiments.

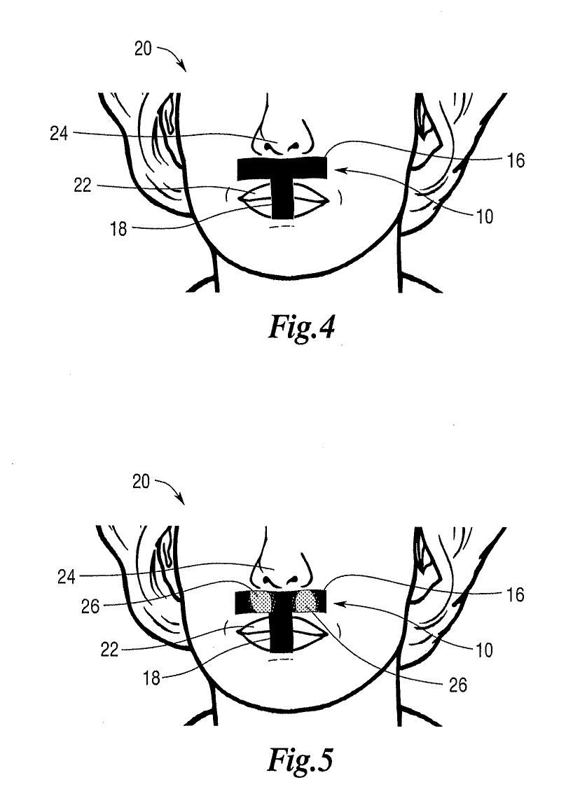

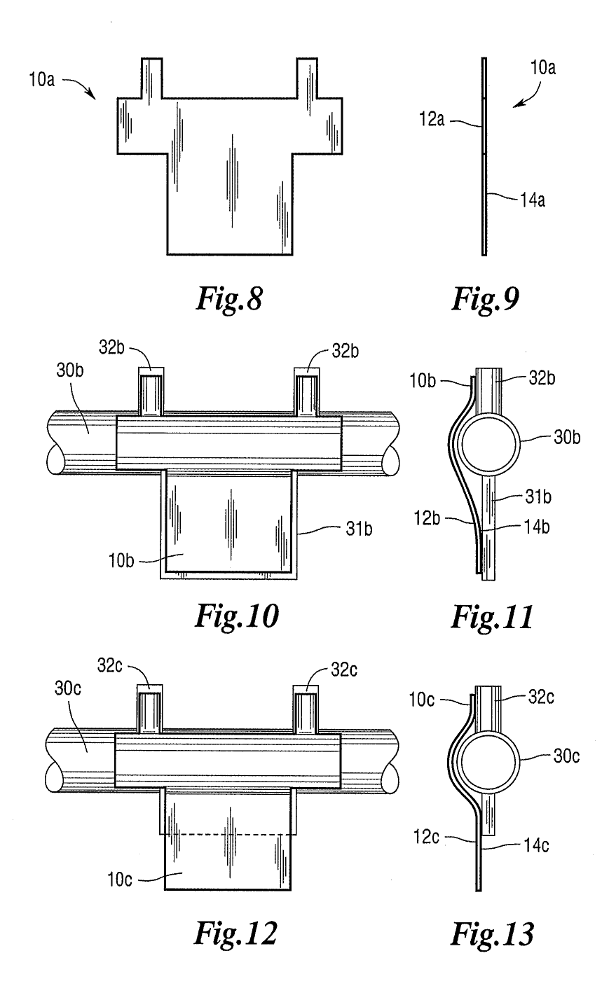

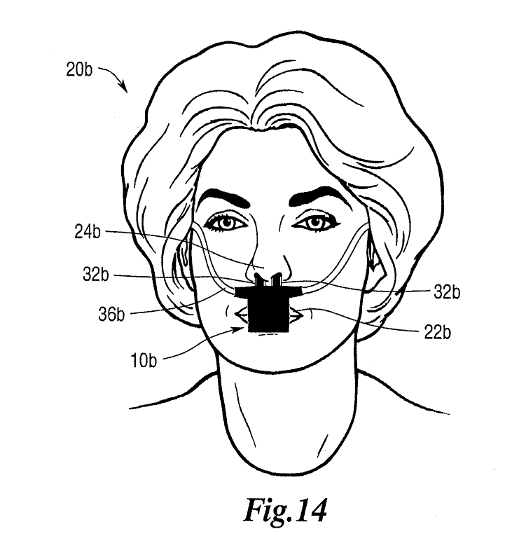

To illustrate these embodiments, FIGS. 8 and 9 show another embodiment of the apparatus in which a layer of adhesive may be used to affix a pre-shaped strip of material 10b to the external surface of a nasal cannula 30b. As shown in FIGS. 10, 11, and 14 the strip of material 10b is shaped to sufficiently cover the nasal cannula 30b. The nasal cannula 30b shown in these figures is a simplified image of a NAZORCAP™ Sampler Model No. 9112-Y which has a multi-perforated oral sampling chamber 31b (the details of which are hidden behind the strip of material 10b and additional features, such as a suspension stem from the sampling chamber, are not shown in the figures) that extends over the mouth. The strip of material 10b has a layer of adhesive on the second side 14b which is used to affix the strip of material 10b to the nasal cannula 30b and to the sampling chamber 31b as shown in the FIGS. 10 and 11. The nasal inlets 32b of the nasal cannula 30b are inserted into a subject's nose 24b such that the thermally sensitive material faces outwards from the subject's 20b face so that it is visible as shown in FIG. 14. Respiration can be monitored as described above for the T-shaped strip of material 10 shown in FIGS. 4 through 6 above.

Loose but secure attachment to the nasal cannula 30b is important because both the first side 12b and the second side 14b of the strip of material 10b needs to be exposed to as much respiratory flow as possible. The thermal properties of some of the materials used in the manufacture of nasal cannula 30b and other respiratory devices cause these devices to retain heat for an extended period of time and not dissipate heat very quickly. This has a negative effect on the ability of the thermally sensitive material to detect temperature variations from respiration. Therefore when securing the strip of material 10b to nasal cannula 30b or other similar devices, it is important to keep the strip of material away from the body of the nasal cannula 30b. Securing the strip of material 10b to create a tent over the nasal cannula 30b as shown in FIGS. 10 and 11 allows respiration to pass about the nasal cannula 30b and be buffeted against the second side 14b of the strip of material 10b and around to the first side 12b. The temperature variance from the respiration is conducted around and through the strip of material 10b and the thermally sensitive material reacts and changes color. As with earlier described embodiments, the actual thermal flow occurs about both the first side 12b and the second side 14b as the temperature variance oscillates between inspiration and expiration.

There are a wide variety nasal cannulas and other respiratory assistance devices available and any of the apparatus disclosed herein could be used on any of them. Another embodiment of nasal cannula 30c shown in FIGS. 12 and 13 is the NAZORCAP™ Sampler Model No. 9801-F, which does not have the oral sampling chamber shown above or a suspension stem thereto, to which a strip of material 10c may be affixed. In nasal cannulas 30c of this type, oral respiration is easier to detect as there would be nothing between the subject's mouth and the strip of material 10c. As with the earlier disclosed embodiment, the strip of material 10c is affixed to form a tent around the portion of the nasal cannula 30c under the subject's nose but hangs down past the body of the nasal cannula 30c over where the subject's mouth would be. Therefore oral respiration is not blocked from reaching the strip of material 10c by the nasal cannula 30c.

In other embodiments, the thermally sensitive material may also be permanently affixed on a visible surface of or incorporated into respiratory assistance devices through a variety of methods during the manufacturing process. The thermally sensitive material could be sprayed or painted onto a surface of the respiratory assistance devices, incorporated into the molding or fabrication of the respiratory assistance devices, or integrally formed into at least one visible surface of the respiratory assistance devices. Preferably, these respiratory assistance devices would be manufactured so that the thermally sensitive material would be readily visible to nearby observers when the respiratory assistance devices are used.

This invention has been described with reference to several preferred embodiments. Many modifications and alterations will occur to others upon reading and understanding the preceding specification. It is intended that the invention be construed as including all such alterations and modifications in so far as they come within the scope of the appended claims or the equivalents of these claims.

What is claimed is:

1. An apparatus for visually determining whether respiration is occurring comprising:
a strip of material having a first side and a second side; and
said first side having a layer of thermally sensitive material which undergoes a color change when exposed to a temperature variance caused by a subject's respiration.

2. The apparatus of claim 1 further comprising said second side having a layer of adhesive.

3. The apparatus of claim 1 further comprising said strip of material being paper, plastic, metal, cloth, thermoplastic polymers, silastic thermopolymers, or polyurethanes.

4. The apparatus of claim 1 further comprising said thermally sensitive material is a thermochromic liquid crystal compound.

5. The apparatus of claim 1 further comprising said thermally sensitive material is selected to be reactive within a temperature range between 27 and 32 degrees Celsius.

6. The apparatus of claim 1 further comprising said thermally sensitive material is selected to be colorimetrically active within a selected temperature range.

7. The apparatus of claim 1 further comprising said strip of material being shaped like a T.

8. The apparatus of claim 1 further comprising said strip of material sized and shaped to fit under the subject's nose and extend over the subject's lip.

9. The apparatus of claim 1 further comprising
said second side having a layer of adhesive; and
said strip of material is adhered to the vicinity of the subject's upper lip with said adhesive.

10. The apparatus of claim 1 further comprising
said second side having a layer of adhesive; and
said strip of material is adhered to a visible surface of a respiratory assistance device with said adhesive.

11. The apparatus of claim 1 further comprising
said second side having a layer of adhesive;
said strip of material is adhered to a visible surface of a respiratory assistance device with said adhesive; and
said respiratory assistance device is a nasal cannula, an oxygen delivery device, a ventilatory assistance device, or an endotracheal tube.

12. An apparatus for visually determining whether respiration is occurring comprising:
a strip of material having a first side and a second side;
said first side having a layer of thermally sensitive material;
said second side being permanently affixed to a respiratory assistance device; and
said layer of thermally sensitive material undergoes a color change when exposed to a temperature variance caused by a subject's respiration.

13. The apparatus of claim 12 further comprising said strip of material being paper, plastic, metal, cloth, thermoplastic polymers, silastic thermopolymers, or polyurethanes.

14. The apparatus of claim 12 further comprising said thermally sensitive material is a thermochromic liquid crystal compound.

15. The apparatus of claim 12 further comprising said thermally sensitive material is selected to be reactive within a temperature range between 27 and 32 degrees Celsius.

16. The apparatus of claim 12 further comprising said thermally sensitive material is selected to be colorimetrically active within a selected temperature range.

17. The apparatus of claim 12 in which said respiratory assistance device is a nasal cannula, an oxygen delivery device, a ventilatory assistance device, or an endotracheal tube.

18. A method for visually determining whether respiration is occurring using a strip of material having a first side and a second side in which the first side has a layer of thermally sensitive material which undergoes a color change when exposed to a temperature variance, comprising:
placing the strip of material so that it is sufficiently exposed to the expected region of respiration of a subject; and
observing whether the thermally sensitive material undergoes a color change.

19. The method of claim 18 in which the second side has a layer of adhesive further comprising adhering the strip of material under the subject's nose with the second side.

20. The method of claim 18 in which the second side has a layer of adhesive further comprising adhering the strip of material to a respiratory assistance device with the second side.

21. The method of claim 18 in which the strip of material sized and shaped to fit under the subject's nose and extend over the subject's lip further comprising placing the strip of material such that the strip of material is located under the subject's nose and extends over the subject's lip.

22. A respiratory assistance device for the delivery and collection of respired gases comprising:
a layer of thermally sensitive material which undergoes a color change when exposed to a temperature variance caused by a subject's respiration, located such that when said respiratory assistance device is installed on the subject, said thermally sensitive material is located immediately below the subject's nose and extends over the subject's mouth.

23. The respiratory assistance device of claim 22 in which said thermally sensitive material is a thermochromic liquid crystal compound.

24. The respiratory assistance device of claim 22 further comprising said thermally sensitive material is selected to be reactive within a temperature range between 27 and 32 degrees Celsius.

25. The respiratory assistance device of claim 22 further comprising said thermally sensitive material is selected to be colorimetrically active within a selected temperature range.

26. The respiratory assistance device of claim 22 in which said thermally sensitive material is sprayed or painted onto said respiratory assistance device.

27. The respiratory assistance device of claim 22 in which said thermally sensitive material is integrally formed into at least one visible surface of said respiratory assistance device.

28. The respiratory assistance device of claim 22 in which said thermally sensitive material is incorporated into the manufacturing of said respiratory assistance device.

29. The respiratory assistance device of claim 22 in which said respiratory assistance device is a nasal cannula, an oxygen delivery device, a ventilatory assistance device, or an endotracheal tube.
